(19) United States
(12) Patent Application Publication
(10) Pub. No.: US 2013/0102064 A1
(43) Pub. Date: Apr. 25, 2013

(54) RT-PCR CHIP WITH OPTICAL DETECTION

(71) Applicant: STMicroelectronics S.r.l., Agrate Brianza (IT)

(72) Inventors: Enzo FONTANA, Catania (IT); Antonino SCUDERI, Misterbianco (IT)

(73) Assignee: STMicroelectronics S.r.l., Agrate Brianza (IT)

(21) Appl. No.: 13/654,264

(22) Filed: Oct. 17, 2012

(30) Foreign Application Priority Data
Oct. 19, 2011 (IT) MI2011A001893

(51) Int. Cl. C12Q 1/68

(52) U.S. Cl. CPC C12Q 1/686
USPC 435/287.2

(57) ABSTRACT

An apparatus (100) for performing thermal cycles has a frame (131-134, 137, 147) enclosing a thermal chamber (110) laterally delimited by delimitation walls (103, 154a, 154b) and configured so as to be delimited at the bottom by a reaction holder (104) carrying a plurality of reaction chambers (107) designed to receive chemical reaction substances. A lid (105), of transparent material, is fixed to the frame and delimits the thermal chamber at the top. A source of light radiation (165) is arranged outside the thermal chamber (110) facing the lid (115) and is configured to generate an excitation light radiation. A detector of light radiation (166) is arranged outside the thermal chamber facing the lid and is configured to collect a light radiation emitted in use by the reaction chambers (107). A processor (171) is connected to the detector of light radiation (166) and is configured to detect, in use, a feature of the light radiation emitted.

### PRIOR RELATED APPLICATIONS

This invention claims priority to Italian application MI2011A001893, filed on Oct. 19, 2011, and incorporated by reference in its entirety herein.

### FEDERALLY SPONSORED RESEARCH STATEMENT

Not applicable.

### REFERENCE TO MICROFICHE APPENDIX

Not applicable.

### FIELD OF THE INVENTION

The present invention relates to a diagnostic apparatus, in particular for performing thermo-cycling operations during an RT-PCR (reverse-transcription polymerase-chain reaction), with optical detection.

### BACKGROUND OF THE INVENTION

As is known, use of diagnostic apparatuses operating on small amounts of specimens is increasingly widespread since they advantageously improve the reliability of the assay, reduce the volume thereof, and thus reduce the time required for this activity, as well as the corresponding costs.

Known devices basically comprise a solid substrate, immobilizing particular receptors, such as, for example, biomolecules (DNA, RNA, proteins, antigens, antibodies, haptens, sugars, etc.) or chemical species, or micro-organisms or parts thereof (bacteria, viruses, spores, cells, organelles, etc.). “Receptors” mean herein any member of a pair or multiple of elements that may bind together (binding pair) so that the receptor binds or reacts with, and thus detects, its own binding mate (or binding mates). Herein, receptors include traditional receptors, such as protein receptors and ligands, but also any element designed to interact or mate, such as, for example, lectins, carbohydrates, streptavidins, biotins, proteins, substrates, oligonucleotides, nucleic acids, porphyrins, metal ions, antibodies, antigens, and the like.

According to the optical-detection technique, when these receptors are arranged in direct contact with a specimen to be analysed, the presence in this specimen of molecules able to mate or interact with the receptor activates specific markers, for example fluorescent markers, which, when excited with a light radiation at a first wavelength, emit light radiation having a second wavelength different from the first wavelength.

Known fluorescence diagnostic devices comprise a compatible layer having a surface that is functionalized so as to form detection areas comprising receptors having the specific markers.

There are many different ways for preparing tests that involve optical signals. For example, a common three-component binding assay uses a first immobilization, on a solid substrate, of an antibody that may mate with an antigen in a specimen solution. Binding with the antigen is then detected using a second antibody, which binds to a different epitope of the same antigen and has a fluorescent label attached thereto. Thus, the amount of fluorescence is correlated to the amount of the antigens in the specimen.

Another solution comprises immobilization on the substrate or the use of a solution of an oligonucleotide probe that is then hybridized with complementary DNA or cDNA or mRNA in the specimen, and the double-strand nucleic acid may be detected with an intercalating dye, such as, for example, ethidium bromide.

According to another solution, two fluorescent markers are brought into strict proximity in the assay, and quenching of a marker is measured in assays based upon fluorescence resonance energy transfer (FRET).

Alternatively, binding of heavy metals with fluorophores may also be detected by means of fluorescent dyes.

Various apparatuses have been proposed having an active or passive approach for detection of the optical signal, irrespective of the treatment performed on the assays and of the technique of generation of the optical signal.

In these apparatuses, the light radiation is collected by a detector, such as, for example, a photodetector of a CCD (charge-coupled device) type or of a CMOS type sensitive to the wavelength of the emitted light radiation, in which the light intensity or its variation is a function of the amount of specific markers activated in the assay, and thus of the amount of detected molecules or biomolecules.

In the case of analysis with qRT-PCR (quantitative reverse-transcriptase polymerase-chain reaction), the apparatus generates thermal cycles (for example, at 60° C., 72° C., and 90° C.) for amplification of the sought target molecule and immediately supplies an accurate quantitative estimate thereof.

In order to perform correctly the process of amplification, the thermal cycles have to occur at controlled and uniform temperature within an area referred to hereinafter also as “reaction chamber”. For example, FIG. 1b shows a portion of an apparatus 1 for performing diagnostic analyses including a reaction zone 2 having guides 3 wherein a holder 4 is inserted and has a heating device 6.

The area above the holder 4 forms a thermal chamber 10 facing light-emitter elements 11, for example LEDs, and a detector 12, for example a CCD detector.

In addition, the apparatus 1 has a ventilating unit 8, for example comprising a fan arranged underneath the reaction zone 2.

An electronic device (not shown) controls the supply of current to the heating device 6 and the activation of the ventilating unit 8 so as to obtain the desired thermal cycles.

The holder 4 (see also FIG. 2 where the holder is shown longitudinally, rotated by 90° with respect to FIG. 1B) is here formed by a moulded body, for example, of suitable biocompatible transparent plastic (for example, polycarbonate) and has a parallelepipedal shape having a bottom on which the heating device 6 is fixed. On the top side, the holder 4 has a series of chambers 7 open at the top and designed to contain the reagents and the reaction products.

As shown in FIGS. 2 and 3, in the example, the heating device 6 is formed by a die, comprising a substrate 15 of semiconductor material having, on one side thereof, a coil 16 of conductive material, for example aluminium or other metal, which extends throughout the area of the chambers 7. The coil 16 is connected to pads 17. Other types of holder are, however, possible, for example without chambers 7; in this case the chambers are formed directly in the die on the back of the heating device.

With the structure shown, it is not possible to obtain a high thermal uniformity in the reaction zone 2. In fact, as shown in the graph of FIG. 1a, which illustrates the thermal profile in the reaction zone 2 along the axis Y, the temperature, which is close to the value TR of the surrounding environment at a distance from the holder 4, increases rapidly to the heating value TH in proximity of the heating device 6, and then drops gradually within the thermal chamber 10, increasing until reaching a value close to the ambient temperature TR above the thermal chamber 10.

In particular, tests conducted by the applicant have shown that, because of dissipation, a thermal gradient exists, within the reaction zone 2, of approximately 10-15° C.

This is disadvantageous since it may jeopardize the correctness of execution of the reactions and thus of the obtained diagnostic result.

Thus, what is needed in the art is a semiconductor based design that eliminates or at least substantially reduces the temperature differential, and provides a more uniform heating platform that can be used in various assays, especially amplification based assays and other temperature sensitive reactions.

### SUMMARY OF THE INVENTION

The aim of the present invention is to provide a diagnostic apparatus that provides a higher thermal uniformity in the reaction zone.

According to the present invention, a diagnostic apparatus is provided, defined as follows:

An apparatus for performing thermal cycles, comprising a frame; a thermal chamber laterally delimited by delimitation walls and configured to be downwardly delimited by a reaction support carrying a plurality of reaction chambers intended to accommodate reaction chemicals; a lid of a transparent material, upwardly delimiting the thermal chamber; a light radiation source, arranged externally to the thermal chamber so as to face the lid and configured to generate an excitation light radiation; a light radiation detector, arranged externally to the thermal chamber so as to face the lid and configured to collect light radiation emitted in use by the reaction chambers; and a processing element, coupled to the light radiation detector and configured to detect, in use, a feature of the emitted light radiation.

In another embodiment, the invention is an apparatus for performing thermal cycles, comprising a parallelepipedal frame, a thermal chamber inside said frame and laterally delimited by side walls, downwardly delimited by a reaction support, and upwardly delimited by a transparent lid. The reaction support has a first heater facing said thermal chamber, and is configured to receive one or more reaction chambers thereon or therein. The lid has a second heater facing said thermal chamber. One or both of said lid and said reaction support is also outfitted with a thermal sensor. The thermal chamber also has air flow paths above and below it (and inside the frame), and one or more fans to draw air along said air flow paths. The thermal sensor(s), heaters and fan(s) are each operably coupled to a processor for controlling the heaters and fan(s) and thus controlling and providing a uniform temperature throughout the chamber. Also inside the frame is a light radiation source, arranged externally to the thermal chamber so as to face the lid and configured to generate an excitation light radiation, a light radiation detector, arranged externally to the thermal chamber so as to face the lid and configured to collect light radiation emitted in use by the reaction chambers, and a processing element, coupled to the light radiation detector and configured to detect, in use, an emitted light radiation.

The following abbreviations are used herein:

| ABBREVIATION | TERM |
|---|---|
| ABS | Acrylonitrile butadiene styrene |
| CCD | Charge-coupled device |
| cDNA | Copy DNA |
| CMOS | complementary-symmetry metal-oxide |
| DNA | Deoxyribonucleic acid |
| FRET | Fluorescence resonance energy transfer |
| GaN | Gallium nitride |
| LED | Light emitting diode |
| mRNA | Messenger RNA |
| PCR | Polymerase chain reaction |
| PVC | Polyvinyl chloride |
| qRT-PCR | Quantitiative real time PCR |
| RNA | Ribonucleic acid |

The use of the word “a” or “an” when used in conjunction with the term “comprising” in the claims or the specification means one or more than one, unless the context dictates otherwise.

The term “about” means the stated value plus or minus the margin of error of measurement or plus or minus 10% if no method of measurement is indicated.

The use of the term “or” in the claims is used to mean “and/or” unless explicitly indicated to refer to alternatives only or if the alternatives are mutually exclusive.

The terms “comprise”, “have”, “include” and “contain” (and their variants) are open-ended linking verbs and allow the addition of other elements when used in a claim.

The phrase “consisting of” is closed, and excludes all additional elements.

The phrase “consisting essentially of” excludes additional material elements, but allows the inclusions of non-material elements that do not substantially change the nature of the invention.

### DESCRIPTION OF THE DRAWINGS

For a better understanding of the present invention, a preferred embodiment thereof is now described, purely by way of non-limiting example, with reference to the attached drawings, wherein:

FIG. 1 displays (A) the thermal profile within a known diagnostic apparatus and (B) a side view of a part of the known diagnostic apparatus;

FIG. 2 is a ghost side view of a holder used in the apparatus of FIG. 1;

FIG. 3 is a bottom view of the holder of FIG. 2;

FIG. 4 displays (A) the thermal profile within the reaction zone of the present diagnostic apparatus and (B) a side view of the reaction zone of the present diagnostic apparatus;

FIG. 5 is a perspective bottom view of the lid in the reaction zone of FIG. 4;

FIG. 6 is an enlarged cross-section and perspective view of a portion of the lid;

FIGS. 7A-7E are different embodiments of the heater arranged on the lid of FIG. 5;

FIG. 8 is a cross-section of the present apparatus, taken along the plane of section VIII-VIII of FIG. 9;

FIG. 9 is a longitudinal section of the present apparatus, taken along plane IX-IX of FIG. 8, including an expanded view of the fan and fan compartment for detail.

FIG. 10 is a block diagram of the present apparatus.

### DESCRIPTION OF THE INVENTION

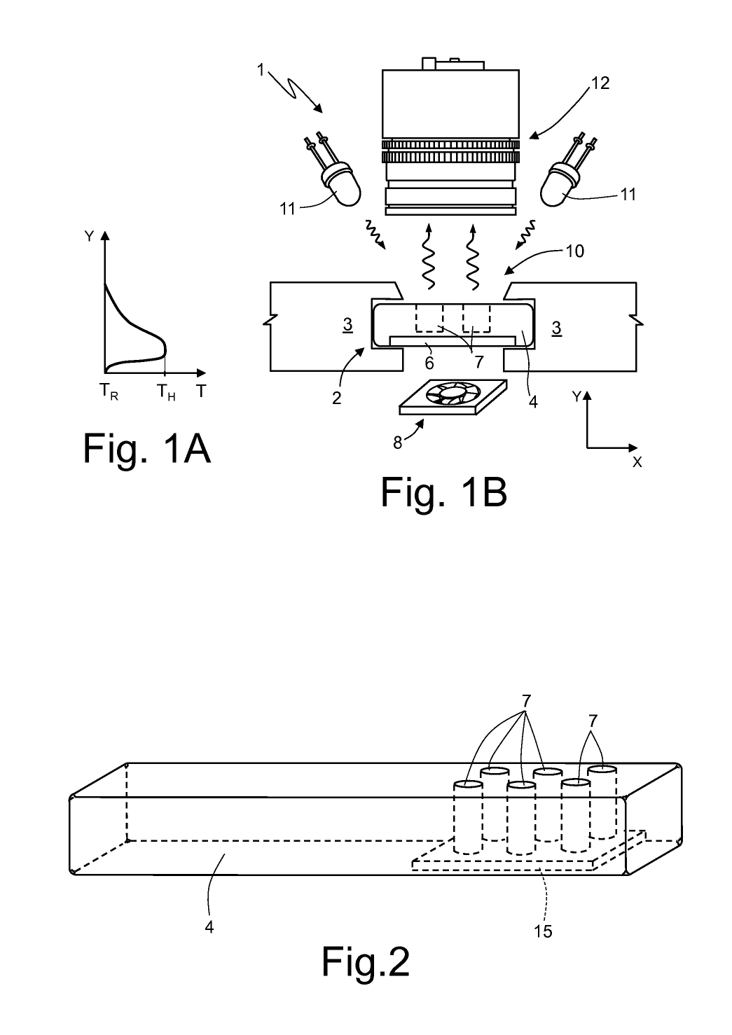

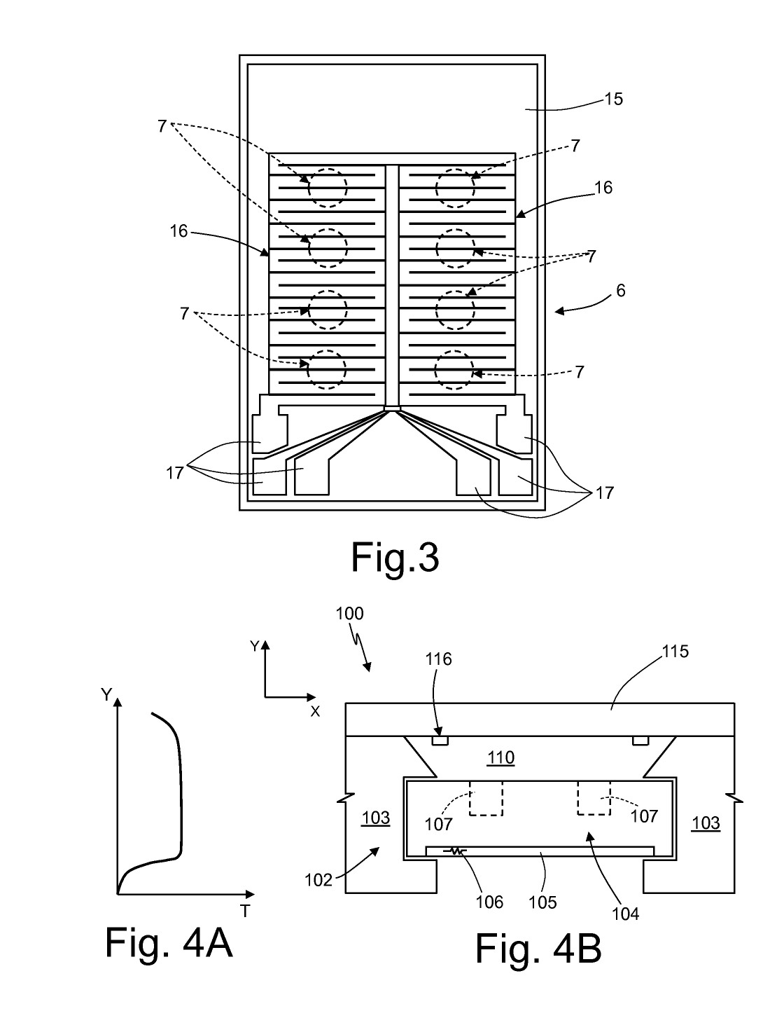

FIG. 4B shows a portion of an apparatus 100 for performing diagnostic analyses in order to detect the presence and amount of target molecules using the qRT-PCR technique referred to above. In particular, FIG. 4B shows a reaction zone 102 including guides 103 for a holder 104 similar to the one described with reference to FIGS. 1-2. In particular, the holder 104 has a first heating device 106 formed in a die 105 and forms a series of reaction chambers 107 (shown in ghost view and open on the top side) designed to contain the reagents and the reaction products and/or test-tubes. Alternatively, the first heating device 106 may be molded directly on the bottom or on a side of the holder 104.

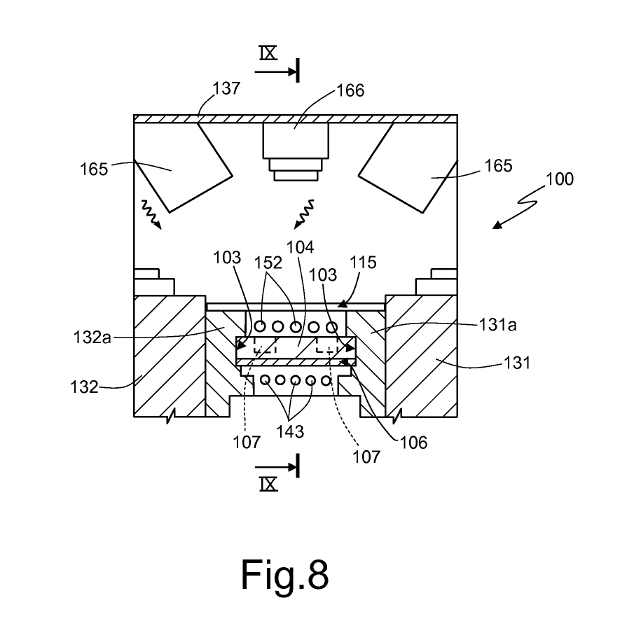

The area above the holder 104 here forms a thermal chamber 110 closed at the top by a lid 115. The lid 115, of a generally rectangular shape, is transparent to the light of emitter elements 165 (FIG. 8) and to the fluorescent light emitted during the reaction so as to enable collection via a detector 166 (also visible in FIG. 8), arranged above the thermal chamber 110. In particular, the lid 115 may be glass, polycarbonate, polyacrylate, GaN or other transparent material to enable passage of the radiation emitted during the reaction, guaranteeing thermal tightness of the underlying reaction zone 102.

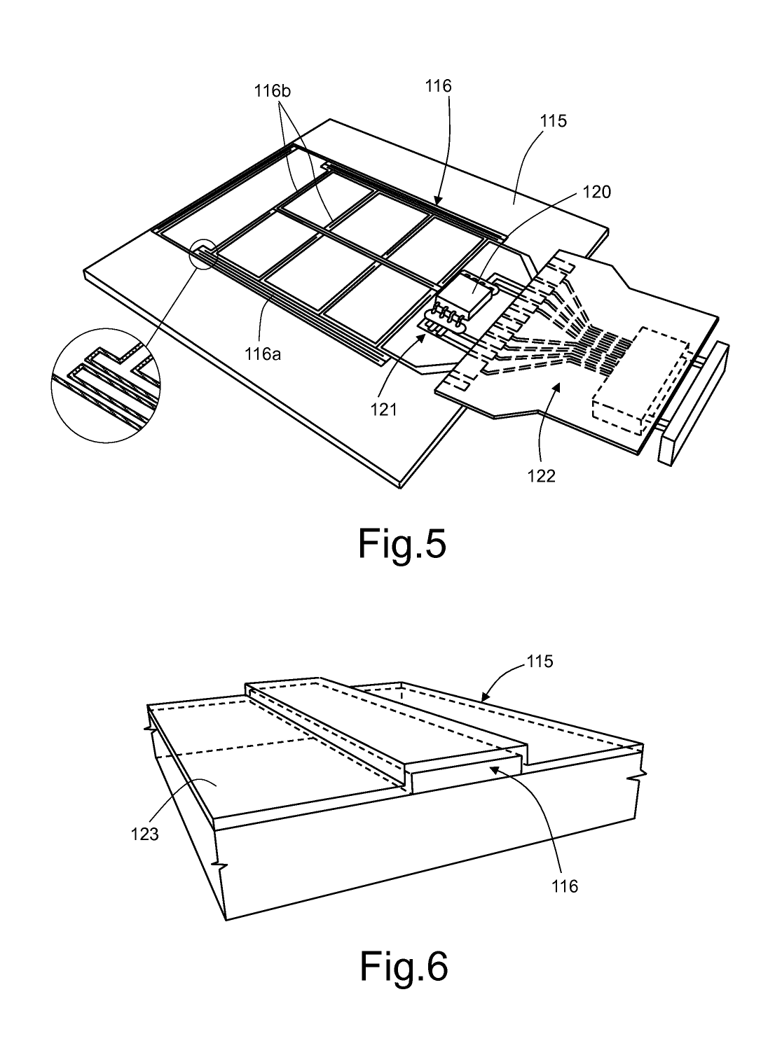

A second heating device 116 is arranged on the lid 115, on the inside surface facing the holder 104 (see also FIG. 5), and is shown in greater detail in FIGS. 5-7.

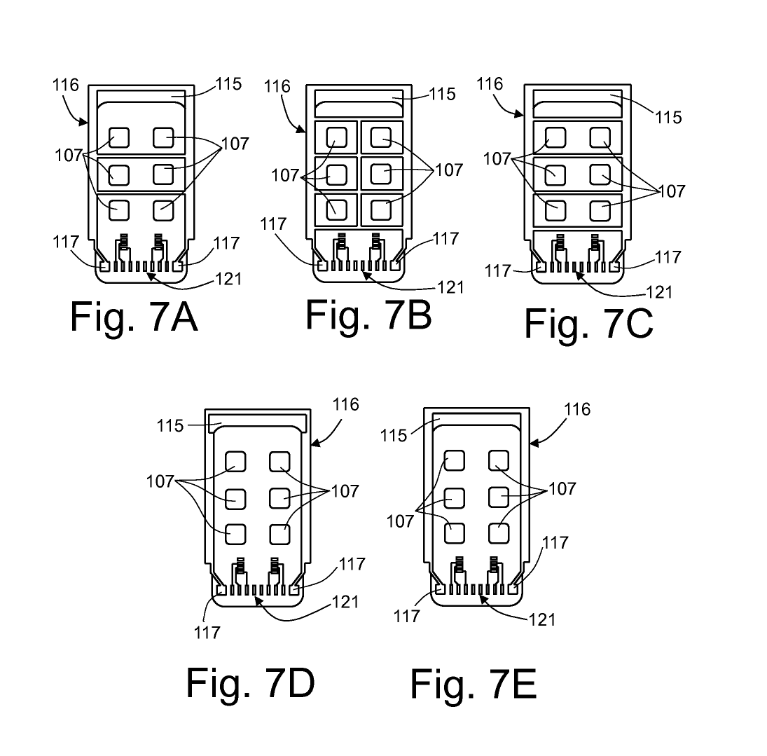

In particular, the second heating device 116 is formed by a conductive path, for example a metal layer, such as Al, Cu or Mo, of large thickness, optimized for a high current capacity (e.g., 2-3 μm able to carry a current comprised between 0.5 A and 2 A) and may have different shapes, shown e.g. in FIGS. 7A-7E. The conductive path generally has a peripheral portion 116a (FIGS. 5, 7A-7E) extending in a direction parallel to three sides of the lid 115 and is connected to pads 117. The peripheral portion 116a may possibly be connected to a plurality of intermediate portions 116b, parallel to and/or crossing one another (FIG. 5, 7A-7C) and passing through the area of the lid 115, preferably so as to lie in the space between one reaction chamber 107 and another, so as not to interfere with the radiation emitted during PCR. In turn, both the peripheral portion 116a and the intermediate portions 116b may be formed completely or partially as coils, as represented in the enlarged detail of FIG. 5.

In order to facilitate initial setup and driving, the first and second heating devices 106, 116 may have the same nominal value of resistance (for example 15 to 24 Ω).

Furthermore, a temperature sensor 120 may be fixed on the lid 115 (FIG. 5), comprises a separate die connected to its own pads 121, and able to measure the temperature in the thermal chamber 110 and to supply a signal that may be used for a temperature feedback control.

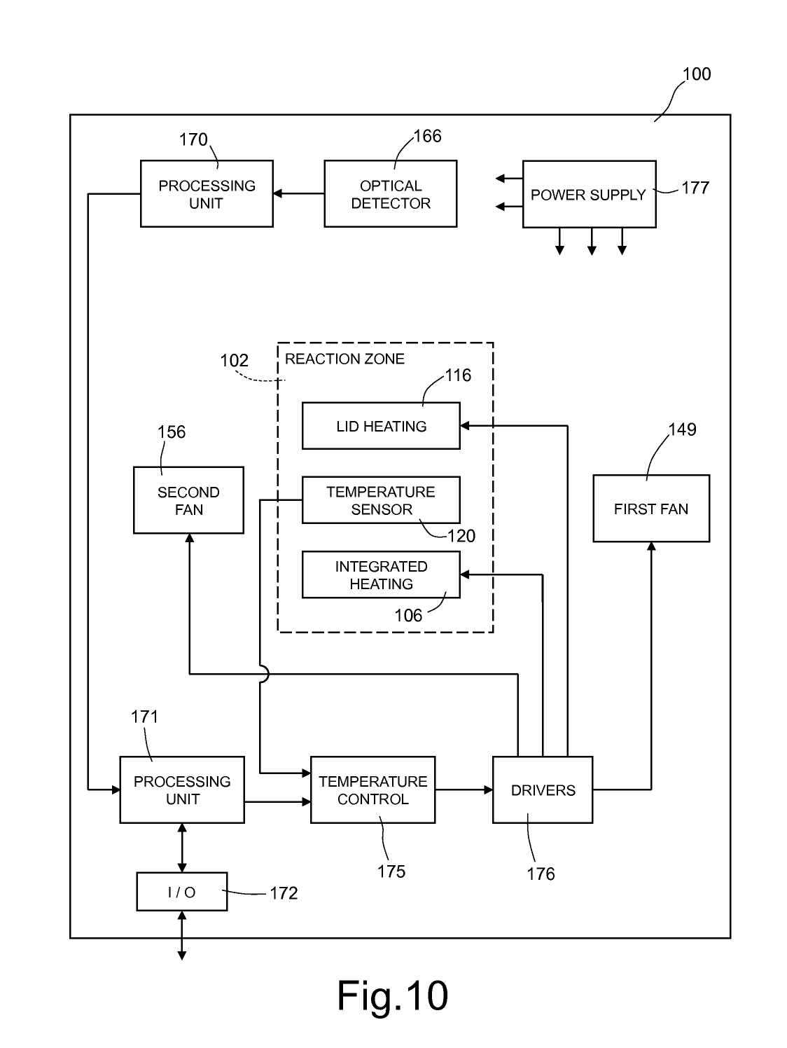

The pads 117, 121 of the second heating device 116 and of the sensor 120 are connected to a connector 122, in turn connected to a control unit of the apparatus 100, as described hereinafter with reference to FIG. 10.

As shown in FIG. 6, the second heating device 116 may be covered by a passivation layer 123 forming a shielding for the metal paths 116a, 116b. For example, the passivation layer 123 may be silicon oxide or silicon nitride with a thickness of approximately 1 μm, and has the function of eliminating possible reflections of the light emitted during the reaction caused by the lid 115, in addition to protecting the metal paths of the second heating device 116.

In this way (see FIG. 4B), the area comprising the thermal chamber 110 and the holder 104 up to the first heating device 106 may be kept at a substantially uniform temperature, as represented in FIG. 4A, thanks to the heating supplied from below by the first heating device 106 and from above by the second heating device 116.

The lid 115 may be manufactured from a plate, for example of glass, having a much greater area than the lid 115, printed with the metal paths 116a, 116b (at the same time forming a number of heating devices 116 for a plurality of lids 115) and covered with the passivation layer 123. The glass plate may then be scored to separate the individual lids 115; the temperature sensor 120 and the connector 122 are mounted on each lid 115 via e.g., conductive paste, glue, or other means, and the lid 115 is mounted on the apparatus 100. All the steps are preferably of a dry type so as to prevent any surface roughness.

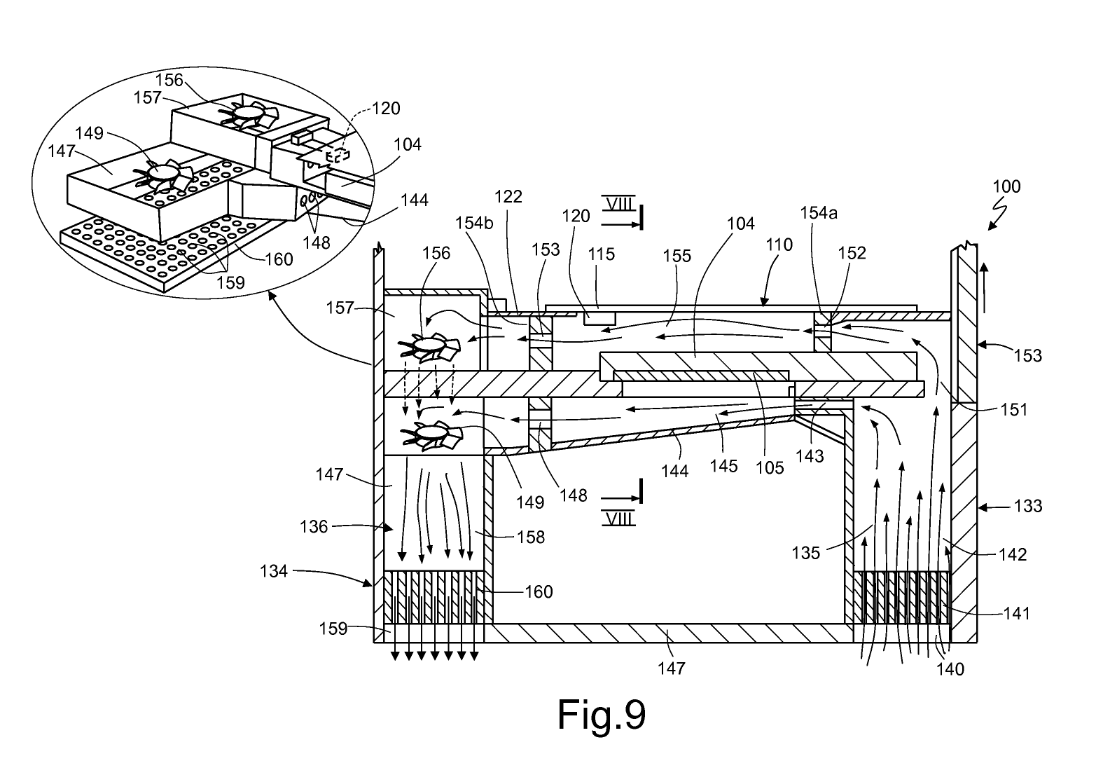

In order to optimize the heating and cooling steps provided for performing the reaction, for example PCR or qRT-PCR, the shown apparatus 100 has a double cooling circuit, as shown in detail in FIGS. 8 and 9, which represent two sections in mutually perpendicular planes.

In detail, the apparatus 100 has a frame defining a closed structure, of a parallelepipedal shape, including a first and a second delimitation walls 131, 132 on the transverse sides (FIG. 8), a first and a second columns 133, 134, on the longitudinal sides (FIG. 9), a bottom 147, and a roof 137. In this way, the walls 131, 132, the columns 133, 134 (in particular with the walls 154a, 154b described hereinafter), the lid 115 and the holder 104 delimit the thermal chamber 110 where the temperature is uniform in a vertical direction (direction Y in FIG. 4B).

Light-emitter elements 165, for example LEDs, and a detector 166, for example a CCD detector, are arranged above the thermal chamber 110, within the frame of apparatus 100.

The delimitation walls 131, 132 define the guides 103 and, together with the columns 133, 134, form resting structures for the lid 105.

Advantageously, the portions 131a, 132a of the delimitation walls 131, 132 that form the guides 103, are formed by rails that may be removed from the rest of the delimitation walls (and are fixed, for example, with screws or other releasable constraint means, such as a ledge they can slide onto or even just a friction fit) so that they may be replaced at each reaction (disposable rails). The rails 131a, 132a are of isothermal material, such as ABS or PVC so as to favor maintenance of a uniform temperature within the reaction zone 102. Further, since the rails can be removed after each reaction and disposed of, this serves to prevent even accidental contamination of the reagents within the chambers in successive analyses.

The columns 133, 134 define internally a double path for the cooling air, including an inlet path 135, a pair of cooling paths 145, 155, and an outlet, common, path 136. In detail, as shown in FIG. 9, the inlet path 135 is formed by an inlet opening 140 on the bottom 147 of the first column 133, by a first grid 141 above the inlet opening 140, and by an inlet chamber 142 above the first grid 141. The first grid 141, as the other grids indicated hereinafter, is formed for example by a mesh or a porous filter so as to prevent suction or in general inlet of dust, in particular particles of large dimensions. Immediately under the reaction zone 102 (FIG. 8), towards the inside of the apparatus 100, the first column 133 has a first plurality of openings 143 (see also FIG. 8, only one visible in FIG. 9) where a part of the air is caused to flow under the reaction zone 102 so as to lap the holder 104 at the bottom (thus forming the first cooling path 145). In order to guide the air along the first cooling path 145 (FIG. 9), a deflector 144 extends underneath the reaction zone 102 between the first and second columns 133, 134, in a longitudinal direction with respect to the apparatus 100.

The second column 134 has, above the end of the deflector 144, a second plurality of openings 148 (only one whereof is visible in FIG. 9) where the air in the first cooling path 145 is drawn, by a first fan 149 arranged in a first fan compartment 147, towards an outlet chamber 158 arranged in the second column 134, along the outlet path 136.

In addition, part of the air flowing through the reaction zone 102 (FIG. 8) is deflected so as to travel over the holder 104 at the top (second cooling path 155). To this end, the inlet chamber 142 is connected at the top to a rear area 151 (behind the holder 104 and closed by a sliding door 153) and then to a third plurality of openings 152 (see also FIG. 8) formed in a first top wall 154a and opening onto the thermal chamber 110. A second fan 156 in a second fan compartment 157 in the second column 134 draws the air along the second cooling path 155 and through a fourth plurality of openings 153 (only one visible in FIG. 9) formed in a second top wall 154b near the top end of the second column 134. As may be seen in FIG. 9, in particular in the enlarged detail, the second fan 156 is arranged at a higher level with respect to the first fan 149, but is offset laterally with respect thereto. Here, the first fan 149 is not aligned longitudinally to the first cooling path 145 but is shifted laterally. Alternatively, the second fan 149 may be offset with respect to the second cooling path 155.

In both cases, the air coming from the first and second cooling paths 145, 155 is sucked into the outlet chamber 158 and discharged through an outlet opening 159 in the bottom 147 and through a second grid 160.

The apparatus 100 has an architecture that is represented in the block diagram of FIG. 10. In detail, the detector 166, as a function of the light radiation collected, generates a first electrical signal supplied to a signal-processing unit 170, which, on the basis of the first received electrical signal, supplies to a processing unit 171 a second electrical signal indicating the reaction that takes place in the chambers 107, in qualitative and/or quantitative terms, according to the implemented reaction and the protocol.

The processing unit 171, in addition to supplying the outside world, through one or more input/output units 172, with the information required, supervises thermal control of the reaction. To this end, it is connected to a temperature-control unit 175, which, through own driving circuits 176, controls actuation of the first and second heating devices 106, 116, supplying the currents necessary for their operation (and thus operating as current source), as well as controlling actuation of the first and second fans 149, 156. The temperature-control unit 175 is moreover connected to the temperature sensor 120 to receive the information on the temperature within the reaction zone 102 so as to control execution of the envisaged thermal cycles in a precise way.

A power-supply unit 177 provides the power supplies requested by the various units of the apparatus 100.

Prior to the reaction, the rear door 153 is opened by sliding it upwards and rendering accessible the rear compartment 151 and the area that is to receive the holder 104. Then, after the possible assembly of the guide portions 131a, 132a in the apparatus 100, the holder 104, with the first heating device 106, is inserted in the rear compartment 151, until it comes to stop against a detent arranged near the wall of the first column 133 (FIG. 9). Then, after closing of the door 153, the diagnostic program may be started, under the control of the central processing unit 171, which then, based on the light radiation emitted, supplies the requested diagnostic result.

The shown apparatus 100 is able to maintain a uniform temperature within the reaction zone 102, and in particular within the chambers 107, by virtue of the presence of two heaters (first and second heating devices 106, 116) arranged on the two sides of the holder 104, which create an air cushion and reduce the thermal gradient between the top part and the bottom part of the holder 104.

The presence of two heaters 106, 116, that may be governed and controlled in an independent way above and under the holder 104, enables setting of the boundary conditions, thus rendering the system independent of the external/environmental variations and shielding the inside.

The presence of two cooling paths 145, 155, which generate two independent flows of air that lap the holder 104 at both its top and at the bottom and are controlled independently, enables precise thermal cycles to be performed, with high thermal uniformity, thanks also to the presence of the temperature sensor 120 on the lid 115.

The fact that the guides 103 are made as disposable rails 131a, 132a favors a high uniformity and prevents any contamination, as explained above.

The lid 115 of transparent material, positioned at a certain distance from the chambers 107 where the reaction takes place, provides a thermal discontinuity with the surrounding environment, without interfering with the optical monitoring of the reaction and the collection of the emitted light radiation.

Finally, it is clear that modifications and variations may be made to the apparatus described and illustrated herein, without thereby departing from the scope of the present invention, as defined in the attached claims.

For example, as indicated, the holder 104 may be different, and likewise the members designed to provide the double cooling path may be made in a way different from the represented one. Similarly additional thermal sensors can advantageously be provided, e.g., below the reaction zone, in addition to the one above.

1. An apparatus for performing thermal cycles, comprising:
a frame;
a thermal chamber laterally delimited by delimitation walls and configured to be downwardly delimited by a reaction support carrying a plurality of reaction chambers intended to accommodate reaction chemicals;
a lid of a transparent material, upwardly delimiting the thermal chamber;
a light radiation source, arranged externally to the thermal chamber so as to face the lid and configured to generate an excitation light radiation;
a light radiation detector, arranged externally to the thermal chamber so as to face the lid and configured to collect light radiation emitted in use by the reaction chambers; and
a processing element, coupled to the light radiation detector and configured to detect, in use, a feature of the emitted light radiation.

2. An apparatus according to claim 1, wherein the lid has a heater facing towards the thermal chamber.

3. An apparatus according to claim 2, wherein the heater is formed by at least one metal track coupled to a current source.

4. An apparatus according to claim 2, comprising a transparent shielding layer covering the lid and the heater on the side thereof facing the thermal chamber.

5. An apparatus according to claim 1, comprising a temperature sensor attached to the lid and facing the thermal chamber.

6. An apparatus according to claim 1, wherein the delimitation walls comprise guide regions of an isothermal material and configured so as to guide the reaction support during the insertion and to laterally hold the reaction support.

7. An apparatus according to claim 6, wherein the guide regions comprise removable, disposable rails.

8. An apparatus according to claim 1, comprising a first and a second cooling circuits including a first and, respectively, a second ventilation unit, wherein the first cooling circuit further includes a lower cooling chamber extending under the thermal chamber and coupled to the first ventilation unit and the second cooling circuit further includes the thermal chamber coupled with the second ventilation unit.

9. An apparatus according to claim 8, wherein the first and the second ventilation units are formed by sucking fans accommodated in a first and respectively a second fan compartments.

10. An apparatus according to claim 9, comprising a first and a second columns extending on opposite sides of the thermal chamber in a longitudinal direction, the first column forming an air inlet chamber coupled with an exterior of the apparatus and the second column forming an air outlet chamber coupled with the first and the second fan compartments and with an exterior of the apparatus, the air inlet chamber being further coupled to the lower cooling chamber through first connection openings and to the thermal chamber via a compartment and via through openings extending in one of the delimitation walls.

11. An apparatus according to claim 9, wherein the first fan compartment is arranged laterally offset with respect to the second fan compartment.

12. An apparatus according to claim 8, comprising a heater, a temperature sensor, a thermal control unit coupled to the heater, to the first and the second ventilation units, and to the temperature sensor for controlling the temperature within the thermal chamber.

13. An apparatus according to claim 12, wherein the thermal control unit further comprises coupling means with an heating element extending under the reaction chambers in the reaction support.

14. An apparatus for performing thermal cycles, comprising:
a parallelepipedal frame;
a thermal chamber inside said frame and laterally delimited by side walls, downwardly delimited by a reaction support, and upwardly delimited by a transparent lid;
said reaction support having a first heater facing said thermal chamber, said reaction support configured to receive one or more reaction chambers thereon;
said lid having a second heater facing said thermal chamber;
one or both of said lid and said reaction support having a thermal sensor;
said thermal chamber having air flow paths above and below said thermal chamber and inside said frame, and one or more fans to draw air along said air flow paths;
said thermal sensor(s) and heaters and fan(s) operably coupled to a processor for controlling said heaters and said fan(s) and thus control temperature inside said thermal chamber;
a light radiation source, arranged externally to the thermal chamber so as to face the lid and configured to generate an excitation light radiation;
a light radiation detector, arranged externally to the thermal chamber so as to face the lid and configured to collect light radiation emitted in use by the reaction chambers;
a processing element, coupled to the light radiation detector and configured to detect, in use, an emitted light radiation.
